(19) United States
(12) Patent Application Publication
(10) Pub. No.: US 2022/0296829 A1
(43) Pub. Date: Sep. 22, 2022

(54) NOSE CONE VENT DESIGN FOR INHALATION DEVICE

(71) Applicant: FUNAI ELECTRIC CO., LTD., Osaka (JP)

(72) Inventors: James D. ANDERSON, JR., Lexington, KY (US); Bruce D. GIBSON, Lexington, KY (US); Brian T. JONES, Lexington, KY (US); Robert W. MILGATE, III, Lexington, KY (US); Tetsuya SHIHARA, Osaka (JP)

(73) Assignee: FUNAI ELECTRIC CO., LTD., Osaka (JP)

(21) Appl. No.: 17/507,931

(22) Filed: Oct. 22, 2021

Related U.S. Application Data
(63) Continuation-in-part of application No. 17/205,280, filed on Mar. 18, 2021.

(51) Int. Cl. A61M 15/08; A61M 15/00

(52) U.S. Cl. CPC A61M 15/08; A61M 15/0025; A61M 2206/10

(57) ABSTRACT

A pharmaceutical drug delivery device. The drug delivery device includes a device body, a fluid outlet nose cone attached to the drug delivery device body, and a fluid jet ejection cartridge containing a liquid pharmaceutical drug is disposed in the drug delivery device body. A fluid ejection head is attached to the fluid jet ejection cartridge and the fluid ejection head is in fluid flow communication with the fluid outlet nose cone. The fluid outlet nose cone has a plurality of air flow channels open to an ambient atmosphere for providing a pressure differential between an inner area of the fluid outlet nose cone adjacent to the fluid ejection head and the ambient atmosphere.

### RELATED APPLICATION

This application is a continuation-in-part of application Ser. No. 17/205,280, filed Mar. 18, 2021, now pending.

### TECHNICAL FIELD

The disclosure is directed to inhalation drug delivery systems and in particular to inhalation devices that have an improved nose cone vent design to reduce fluid flooding of a fluid ejection head.

### BACKGROUND AND SUMMARY

Nasal spray devices have become important methods for delivering drugs to patients. Such nasal spray devices are more convenient to use than the administration of drugs through intravenous (IV) injection. Nasal spray devices also provide higher bioavailability of drugs compared to oral administration of drugs. The absorption of drugs through nasal spray devices is more rapid compared to the absorption of drugs administered orally since drugs delivered by nasal spray devices directly enter the blood stream making their effect more immediate.

FIG. 1 is a cross sectional view, not to scale, of anatomy of a nasal cavity 10 of a person. A portion of the brain 12 is shown above the nasal cavity 10. An olfactory bulb 14 is disposed between the brain 12 and a cribriform plate 16. An olfactory mucosa 18 is below the cribriform plate 16. The nasal cavity also includes a superior turbinate 20, a middle turbinate 22, respiratory mucosa 24 and an inferior turbinate 26. Item 28 represents the palate. Injection of a pharmaceutical drug mist enters the nasal cavity 10 through the nostrils 30 and squamous mucosa 32. In order to achieve proper delivery of drugs to the blood stream using a nasal spray device, the drugs must be delivered to the respiratory mucosa which is highly vascularized. Two areas that are highly vascularized are the olfactory region and the respiratory region. The respiratory region contains turbinates which increase the surface area available for drug absorption.

Conventional methods for delivering drugs via the nasal cavity include medicine droppers, multi-spray bottles with spray tips, single-dose syringes with spray tips, and dry powder systems. Accordingly, conventional drug delivery devices are typically designed to deliver a specific drug to a nasal cavity and each device cannot be adapted for delivering a wide range of drugs via a nasal cavity route. Many of the conventional methods for nasal drug delivery rely on pressurized containers to inject a mist of fluid into the nasal cavity. Accordingly, the drug delivery devices are typically designed for a specific drug and cannot be adapted to administer a different drug.

Despite the availability of a variety of devices for delivering drugs via a nasal cavity route, there remains a need for a nasal drug delivery device that can be adapted to deliver a variety of drugs. One such device is an on-demand fluid jet delivery device. Conventional fluid jet delivery devices operate to eject fluid to a substrate under ambient atmospheric pressure. For a nasal drug delivery device, a predetermine fluid jet and fluid plume is desired in order to deliver of a precise amount of pharmaceutical drug to the user. However, a nose cone of the nasal drug delivery device is inserted into one nostril of a user, the nostril is closed off to the ambient atmosphere. Studies have shown that pulling too much vacuum on nozzles of a fluid jet ejection head will pull fluid through the nozzles. The effect of pulling fluid through the nozzles is worse when the ejection head is activated to eject fluid as a jet stream into a nasal cavity and the user inhales during the jetting operation. When a moderate vacuum is pulled on a nozzle plate of a fluid jet ejection head during a fluid jetting sequence, fluid jetting is disturbed, and a desired fluid plume fails to form resulting in fluid drooling out of the nozzle plate and accumulating on the surface of the nozzle plate.

Another problem associated with using a fluid jet ejector device to administer drugs to a user's nasal cavity is that fluid may dry out on the ejection head between uses and interfere with fluid ejected through fluid nozzles on the ejection head causing misdirection of fluid and misfiring of fluid ejectors. Accordingly, what is needed is an improved nasal applicator that is designed to prevent drooling of fluid from a fluid cartridge having a fluid jet delivery device to eject fluid into a nasal cavity of a user. The device must also prevent fluid from drying out on the ejection head between uses of the device.

Accordingly, what is needed is an improved nasal applicator that is designed to prevent drooling and accumulation of fluid from a fluid cartridge on the ejection head or nozzle plate of a device using a fluid jet ejection head to eject fluid into a nasal cavity of a user.

In view of the foregoing an embodiment of the disclosure provides a pharmaceutical drug delivery device. The drug delivery device includes a device body, a fluid outlet nose cone attached to the drug delivery device body, and a fluid jet ejection cartridge containing a liquid pharmaceutical drug is disposed in the drug delivery device body. A fluid ejection head is attached to the fluid jet ejection cartridge and the fluid ejection head is in fluid flow communication with the fluid outlet nose cone. The fluid outlet nose cone has a plurality of air flow channels open to an ambient atmosphere for providing a pressure differential between an inner area of the fluid outlet nose cone adjacent to the fluid ejection head and the ambient atmosphere.

In another embodiment there is provided a method for reducing a pressure differential on a fluid jet ejection head for a nasal spray device. The method includes providing a fluid outlet nose cone attached to a body for the nasal spray device and a fluid jet ejection cartridge disposed in the body. The fluid jet ejection cartridge contains the fluid ejection head in fluid flow communication with the fluid outlet nose cone and a pharmaceutical drug in the fluid jet ejection cartridge. A plurality of air flow channels open to an ambient atmosphere are included for providing a pressure differential between an inner area of the fluid outlet nose cone adjacent to the fluid ejection head and the ambient atmosphere. The nasal spray device is activated while flowing air through the air flow channels when the fluid outlet nozzle is inserted into the nasal passage of a user thereby preventing fluid from drooling from the fluid ejection head upon inhalation by a user of the nasal spray device.

In another embodiment there is provided a nose cone for a nasal spray device. The nose cone includes a fluid inlet adjacent to a fluid jet ejection head and a fluid outlet for delivery of a pharmaceutical drug to a nasal cavity of a user, wherein the nose cone has a plurality of air flow channels in an exterior surface thereof for providing a pressure differential between the fluid inlet and the ambient atmosphere.

In another embodiment, the pressure differential ranges from about 5 to about 10 kPa at an air flow rate of from about 50 to about 100 L/min.

In another embodiment, the plurality of air flow channels are adjacent to an exterior surface of the fluid outlet nose cone.

In some embodiments, the plurality of air flow channels extend along a long axis of the fluid outlet nose cone.

In some embodiments, the plurality of air flow channels comprise elongate channels in an exterior surface of the fluid outlet nose cone.

In other embodiments, the elongate channels are partially covered by an outer nose cone structure.

In some embodiments, the plurality of air flow channels have a combined air flow area ranging from about from about 8.0 to about 17.5 mm².

In some embodiments, the nose cone includes a twistable closure configured to provide a variable air flow rate ranging from about 50 to about 100 L/min.

An advantage of disclosed embodiments is the provision of a drug delivery device that can be used to provide a reduced pressure differential on a jet ejection head during use of the pharmaceutical device and that can be sealed to maintain a moist environment adjacent to the ejection head when not in use.

### BRIEF DESCRIPTION OF THE DRAWINGS

FIG. 1 is a cross-sectional representation, not to scale, of a portion of a nasal cavity and scull of a person.

FIG. 2 is a perspective view, not to scale of a pharmaceutical drug delivery device according to an embodiment of the disclosure.

FIG. 3 is a schematic view of the drug delivery device of FIG. 2 illustrating a fluid jet and fluid plume from the device during fluid ejection.

FIG. 4 is a partial perspective, cross-sectional view, not to scale, of the drug delivery device of FIG. 2 containing an according to an embodiment of the disclosure.

FIG. 5 is a partial cross-sectional view, not to scale, of insert in the fluid outlet nozzle of the device of FIG. 3.

FIG. 6 is a perspective view, not to scale, of a nose cone according to a first embodiment of the disclosure for the drug delivery device of FIG. 2.

FIG. 7 is a cross-sectional view, not to scale, of the nose cone of FIG. 6.

FIG. 8 is a perspective view, not to scale, of a nose cone according to a second embodiment of the disclosure for the drug delivery device of FIG. 2.

FIG. 9 is a cross-sectional view, not to scale, of the nose cone of FIG. 8.

FIG. 10 is a side view, not to scale, of a nose cone according to a third embodiment of the disclosure.

FIG. 11 is a cross-sectional, perspective view of the nose cone of FIG. 10.

### DETAILED DESCRIPTION

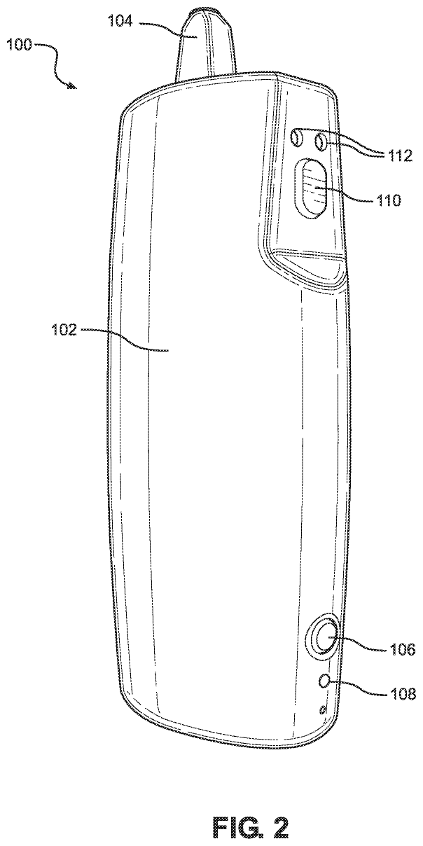

An illustration of a pharmaceutical drug delivery device 100 is illustrated in FIG. 2. The device includes a drug delivery device body 102, having a nose cone 104 attached to the drug delivery device body 102. A power button 106 is provided to activate the drug delivery device as indicated by an LED 108. During use of the device 100, a dispense button 110 is pressed and fluid delivery is indicated by LED's 112. When not in use, a cap may be used to cover the nose cone to prevent fluid from drying out on a fluid jet ejection head used to deliver the pharmaceutical drug to a user. The drug delivery device body 102 also contains a power source and a controller for controlling the ejection of fluid from the fluid jet ejection head.

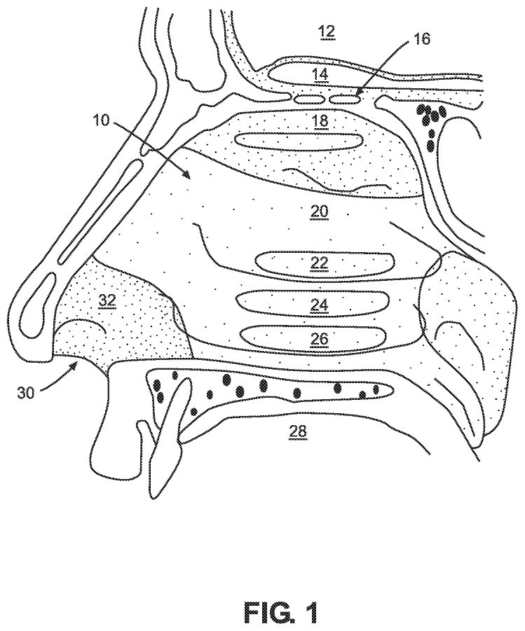

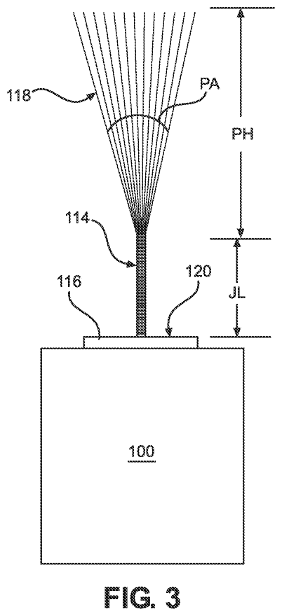

FIG. 3 is a schematic view of the operation of the drug delivery device 100 according to the disclosure. When the device 100 is activated by dispense button 110, a jet of fluid is emitted from the fluid jet ejection head 116 with a length of JL. As the jet of fluid 114 moves further into the nasal cavity a plume of fluid 118 forms having a plume angle of PA and a plume height of PH. Upon inhalation by the user of the drug delivery device, the plume of fluid 118 moves further into the nasal cavity 10 (FIG. 1).

During inhalation, a person's lungs provide a maximum negative lung pressure of −3 kPa for normal breathing. However, deep inhalation causes a much higher negative lung pressure that is about 5 times higher than the negative lung pressure during normal breathing. It is believed that the average maximum inhalation pressure for men is about 9.5 +/−5.1 kPa or a maximum of about 15 kPa. Common breathing flow rates are about 20 l/min for normal breathing, 60 L/min for a typical sniff, and up to about 150 L/min for deep inhalation. Accordingly, sniffing, or deep inhalation can cause a significant negative pressure adjacent to a surface 120 of the ejection head 116. Such negative pressure may cause a disruption of the fluid jet 114 and fluid plume 118 and may cause fluid to drool or leak out of the ejection head 116 and accumulate on the surface 120 of the ejection head. Even a small negative pressure on the surface 120 of the ejection head 116 may cause fluid to leak out and dry on the surface 120 of the ejection head 116. Both dried fluid and fluid accumulation on the surface 120 of the ejection head 116 may significantly disrupt the proper operation of the drug delivery device 100 to provide the desired amount of fluid to specific locations in the nasal cavity 10.

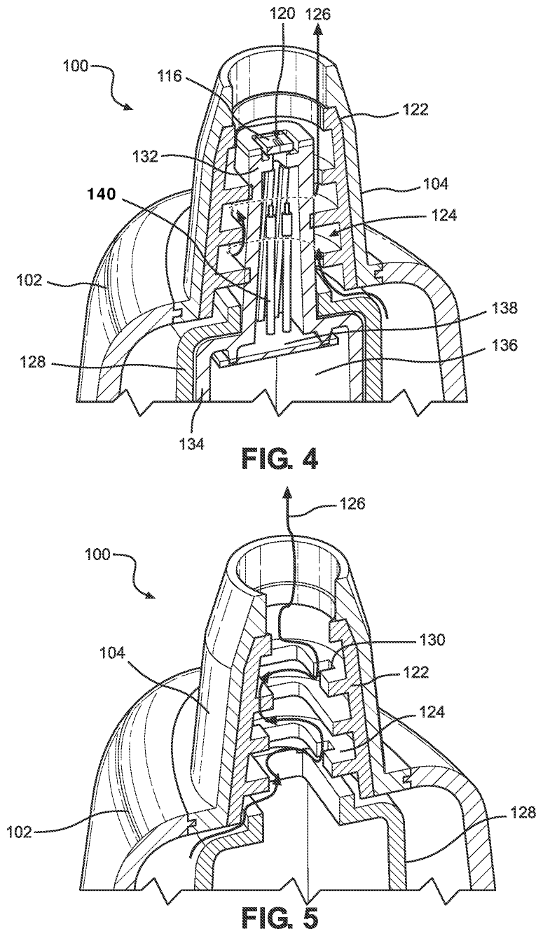

FIGS. 4 and 5 are cross-sectional, perspective views, not to scale of a portion of the drug delivery device 100 having an insert 122 that provides chamber 124 for retaining a volume of moist air therein to prevent drying out of the surface 120 of the ejection head 116. Moist air may enter the device body 102 through openings therein and flow through the insert 122 in the direction of arrow 126 through the insert 122 to provide moist air to the surface 120 of the ejection head 116. The moist air may flow between the device body 102 and a cartridge holder 128 through alternating notches 130 in the insert 122 formed between the insert 122 and an ejection head support structure 132.

The insert 122 for use with the device 100 may be selected from a wide variety of sulfur-free resilient materials such as natural or synthetic rubber, and thermoplastic or thermoset elastomers having a shore A durometer of less than about 60 that are compatible with the fluids being ejected from the ejection head 116. Examples of such materials include, but are not limited to natural rubber, EPDM rubber, and a dynamically vulcanized alloy consisting mostly of fully cured EPDM rubber particles encapsulated in a polypropylene (PP) matrix, available from ExxonMobil under the tradename SANTOPRENE. The insert 122 may be molded and shaped to provide the chambers 124 and alternating notches 130 described above. It will be appreciated that the drug delivery device body 102 of the device 100 is not air-tight and thus provides inlet air flows from a variety of locations such as from the buttons 106 and 110 and any opening provided for inserting the fluid into the drug delivery device body 102.

A fluid jet ejection cartridge 134 containing a pharmaceutical fluid 136 is disposed in the cartridge holder 128. A fluid filter 138 is disposed in the cartridge 134 to filter the fluid flowing through filter tower structures 140 to a fluid jet eject ejection head 116. In some embodiments, the fluid cartridge 134 may also contain a backpressure control device such as a bladder or foam for inducing a backpressure on the fluid jet ejection head 116. The fluid jet ejection head 116 may be selected from any of the conventional types of fluid jet ejection heads, including but not limited to, thermal jet ejection heads, bubble jet ejection heads, piezoelectric jet ejection heads, and the like. Each of the foregoing ejection heads can produce a spray of fluid on demand.

As set forth above, when the nose cone 104 of the device 100 is inserted into the nostril 30 of a user, and the user inhales, a low pressure area is formed adjacent to the surface 120 of the ejection head 116. This low pressure area creates a “pressure differential” between the surface 120 of the ejection head 116 and the fluid in the cartridge 134. The pressure differential can cause unwanted flow or drooling of fluid from the ejection head 116 and may disrupt the fluid jet 114 and fluid plume 118 when the drug delivery device 100 is activated.

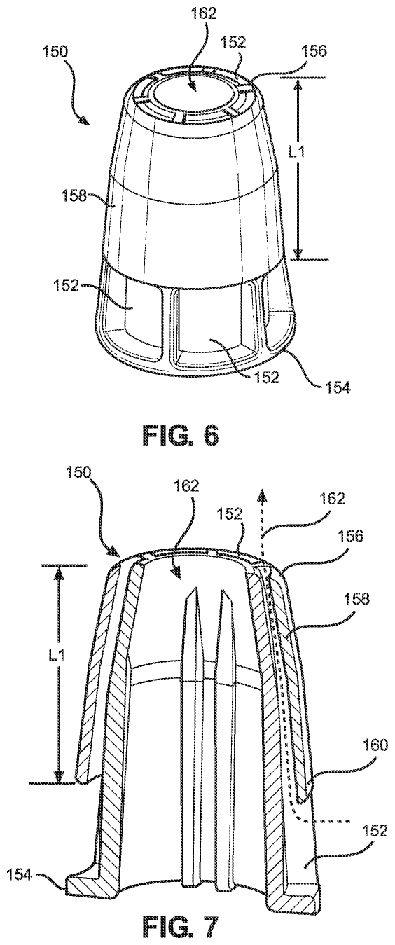

In order to reduce a pressure differential on the surface 120 of the ejection head 116 that would cause leaking or drooling of fluid from the ejection head during inhalation of fluid from the device, modified nose cones are provided in FIGS. 6-11. The nose cones in FIGS. 6-11 portion of the nose cone adjacent to the ejection head 116.

In FIGS. 6 and 7, a modified nose cone 150 is provided that can be used with the insert 122 described above. The nose cone 150 includes relatively wide channels 152 around an outside perimeter of the nose cone 150. The relatively wide channels 152 extend from a bottom portion 154 of the nose cone 150 where the nose cone is attached to the device body 102 to a top portion 156 of the nose cone 150. In order to prevent the nostrils 30 of a user from closing off the channels 152, an upper shroud 158 covers a portion of the wide channels 152 and extends from the top portion 156 to a midway portion 160 of the nose cone 150. A length L1 of the upper shroud 158 is sufficient to prevent the nostril 30 of a user from closing off the wide channels 152 from the atmosphere.

The relatively wide channels 152 provide a pressure differential between an inner area 162 of the nose cone 150 adjacent to the fluid ejection head and the ambient atmosphere of air passing through the channels 152 ranging from about 5 to about 10 kPa at an air flow rate of from about 50 to about 100 L/min through the channels 152. The channels 152 thus allow ambient air to pass through the channels 152 in the direction of arrow 162 into the nasal cavity 10 upon inhalation by the user without providing a negative pressure in the inner area 164 of the nose cone that is sufficient to cause fluid to leak or drool from the ejection head 116.

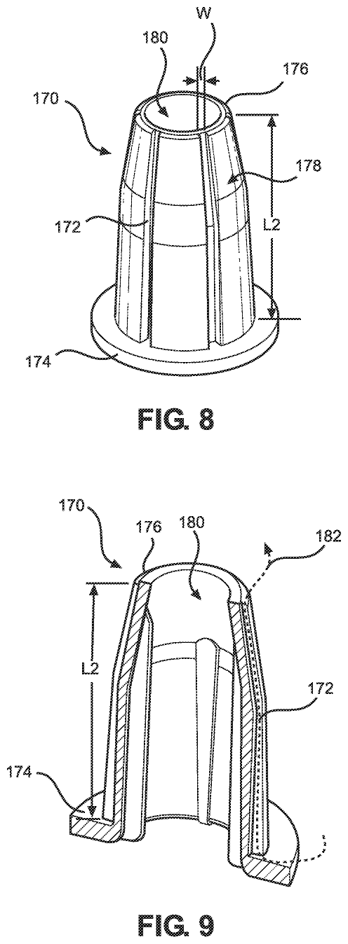

FIGS. 8 and 9 provide an alternative nose cone design that may be used with the insert 122 to reduce the pressure differential adjacent to the ejection head 116. Instead of wide channels 152, the nose cone 170 of FIGS. 8 and 9 includes a plurality of elongate narrow channels 172 that extend axially from a bottom portion 174 to a top portion 176 of the nose cone 170. The elongate channels 172 are formed in an outer surface area 178 of the nose cone 170. Each of the channels has a length L2 and a width W that provides a combined air flow area as shown by arrow 182 that is sufficient to enable a pressure differential between an inner area 180 of the nose cone 170 adjacent to the fluid ejection head and the ambient atmosphere of air passing through the combined channels 172 to range from about 5 to about 10 kPa at an air flow rate of from about 50 to about 100 L/min. The width W of the channels 172 is such that the nostrils 30 of the user cannot close off the channels 172, thus the need for an outer shroud, as in FIGS. 6 and 7 is eliminated.

In order to determine the air flow area for channels 152 and 172 to provide about 50 to about 100 L/min of air flow, the following equation may be used:

$$Q = {C}_{d} {A [ \frac{2}{ρ} Δ p ]}^{\frac{1}{2}} {m}^{3} / s$$

where:
Q=air flow rate (m³/s)
C_{d}=discharge coefficient
ρ=air density (kg/m³⁾
Δp=pressure difference across opening (Pa)
A=area of opening (m²)

### Assuming:

C_{d}=0.63 (typical value)
ρ=1.21 (kg/m³) (typical value)
airflow rate Q=ranging from 50 L/min (0.0008 m³/sec) to 100 L/min (0.0017 m³/s)
maximum pressure differential=15,000 Pa (from limits above),
   the orifice area A range is calculated as follows:

$$A = \frac{Q}{{{C}_{d} [ \frac{2}{ρ} Δ p ]}^{\frac{1}{2}}} \\ A = \frac{0.0008 to 0.0017}{{0.63 [ \frac{2}{1.21} * 15000 ]}^{\frac{1}{2}}} \\ so that \\ A = 0.81 \times {10}^{- 5} to 1.71 \times {10}^{- 5} {m}^{2} .$$

Accordingly, both sets of channels 130 and 172 of FIGS. 6-11 have a combined air flow area ranging from about 8.0 to about 17.5 mm².

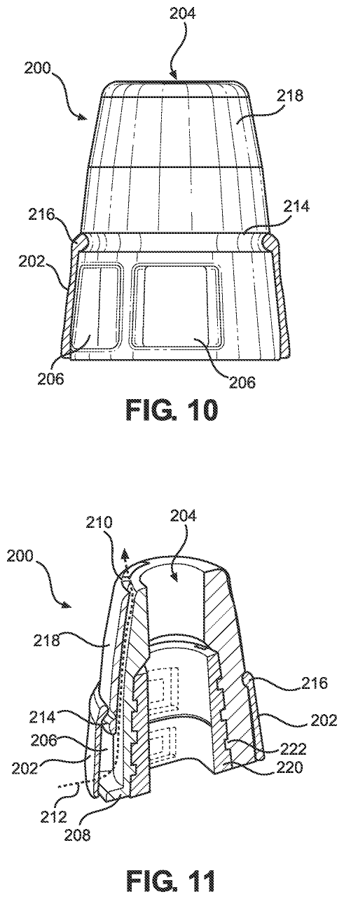

In some embodiments, it may be useful to control the amount of air flow through the nose cone during inhalation. For example, adults may require more ambient air flow through the channels 152 (FIGS. 6-7) to prevent a negative pressure in the inner area 164 of the nose cone that is sufficient to cause fluid to leak or drool from the ejection head 116. Accordingly, a nose cone 200 (FIGS. 10-11) may be provided with a twistable closure 202 to vary the amount of air required to prevent a negative pressure in an inner area 204 of the nose cone. In this embodiments, the nose cone 200 includes two or more wide channels 206 around an outside perimeter of the nose cone 200 for ambient air flow therethrough. The two or more wide channels 206 extend from a bottom portion 208 to a top portion 210 of the nose cone to provide air flow in the direction of arrow 212. As in the previous embodiments, the two or more channels 206 allow air flow into the nasal cavity during inhalation to prevent an unwanted negative pressure on the inner area 204 of the nose cone. The twistable closure 202 is positioned over a lower portion of the nose cone 200, and the nose cone contains a groove 214 for engaging an upper portion 216 of the twistable closure 202. As the twistable closure 202 is rotated around an outside perimeter of the nose cone 200, air flow volume through channels 206 may be changed to increase or decrease the air flow through the channels 206. As in the embodiment of FIGS. 6-7, an upper portion 218 of the nose cone 200 prevents the nostrils of a user from closing off the channels 206 during use. As in the other embodiments, described above, the nose cone 200 also includes an insert 220 that provides chambers 222 for retaining a volume of moist air therein to prevent drying out of the surface 120 of the ejection head 116 when the nose cone is capped.

It is noted that, as used in this specification and the appended claims, the singular forms “a,” “an,” and “the,” include plural referents unless expressly and unequivocally limited to one referent. As used herein, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that can vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques.

While particular embodiments have been described, alternatives, modifications, variations, improvements, and substantial equivalents that are or can be presently unforeseen can arise to applicants or others skilled in the art. Accordingly, the appended claims as filed and as they can be amended are intended to embrace all such alternatives, modifications variations, improvements, and substantial equivalents.

What is claimed is:

1. A pharmaceutical drug delivery device comprising:
a drug delivery device body;
a fluid outlet nose cone attached to the drug delivery device body;
a fluid jet ejection cartridge containing a liquid pharmaceutical drug is disposed in the drug delivery device body, wherein a fluid ejection head is attached to the fluid jet ejection cartridge and the fluid ejection head is in fluid flow communication with the fluid outlet nose cone,
wherein the fluid outlet nose cone comprises a plurality of air flow channels open to an ambient atmosphere for providing a pressure differential between an inner area of the fluid outlet nose cone adjacent to the fluid ejection head and the ambient atmosphere.

2. The pharmaceutical drug delivery device of claim 1, wherein the pressure differential ranges from about 5 to about 10 kPa at an air flow rate of from about 50 to about 100 L/min.

3. The pharmaceutical drug delivery device of claim 1, wherein the plurality of air flow channels are adjacent to an exterior surface of the fluid outlet nose cone.

4. The pharmaceutical drug delivery device of claim 1, wherein the plurality of air flow channels extend along a long axis of the fluid outlet nose cone.

5. The pharmaceutical drug delivery device of claim 1, wherein the plurality of air flow channels comprise elongate channels in an exterior surface of the fluid outlet nose cone.

6. The pharmaceutical drug delivery device of claim 5, wherein the elongate channels are partially covered by an outer nose cone structure.

7. The pharmaceutical drug delivery device of claim 1, wherein the plurality of air flow channels have a combined air flow area ranging from about 8.0 to about 17.5 mm².

8. The pharmaceutical drug delivery device of claim 1, wherein the nose cone further comprises a twistable closure configured to provide a variable air flow rate ranging from about 50 to about 100 L/min.

9. A method for reducing a pressure differential on a fluid jet ejection head for a nasal spray device, the method comprising:
providing a fluid outlet nose cone attached to a body for the nasal spray device; a fluid jet ejection cartridge disposed in the body, the fluid jet ejection cartridge containing the fluid ejection head in fluid flow communication with the fluid outlet nose cone and a pharmaceutical drug in the fluid jet ejection cartridge;
providing a plurality of air flow channels open to an ambient atmosphere for providing a pressure differential between an inner area of the fluid outlet nose cone adjacent to the fluid ejection head and the ambient atmosphere; and
activating the nasal spray device while flowing air through the air flow channels when the fluid outlet nozzle is inserted into the nasal passage of a user thereby preventing fluid from drooling from the fluid ejection head upon inhalation by a user of the nasal spray device.

10. The method of claim 9, wherein the pressure differential ranges from about 5 to about 10 kPa at an air flow rate of from about 50 to about 100 L/min.

11. The method of claim 9, wherein the plurality of air flow channels are adjacent to an exterior surface of the fluid outlet nose cone.

12. The method of claim 9, wherein the plurality of air flow channels extend along a long axis of the fluid outlet nose cone.

13. The method of claim 9, wherein the plurality of air flow channels comprise elongate channels in an exterior surface of the fluid outlet nose cone.

14. The method of claim 13, wherein the elongate channels are partially covered by an outer nose cone structure.

15. The method of claim 9 wherein the plurality of air flow channels have a combined air flow area ranging from about 8.0 to about 17.5 mm².

16. The method of claim 15, wherein the nose cone comprises a twistable closure, further comprising twisting the twistable closure to increase or decrease the combined are flow area.

17. A nose cone for a nasal spray device, comprising a fluid inlet adjacent to a fluid jet ejection head and a fluid outlet for delivery of a pharmaceutical drug to a nasal cavity of a user, wherein the nose cone comprises a plurality of air flow channels in an exterior surface thereof for providing a pressure differential between the fluid inlet and the ambient atmosphere.

18. The nose cone of claim 17, wherein the pressure differential ranges from about 5 to about 10 kPa at an air flow rate of from about 50 to about 100 L/min.

19. The nose cone of claim 17, wherein the plurality of air flow channels extend along a long axis of the nose cone.

20. The nose cone of claim 17, wherein the plurality of air flow channels comprise elongate channels.

21. The nose cone of claim 20, wherein the elongate channels are partially covered by an outer nose cone structure.

22. The nose cone of claim 17, wherein the plurality of air flow channels have a combined air flow area ranging from about 8.0 to about 17.5 mm².

23. The nose cone of claim 17, wherein the nose cone further comprises a twistable closure configured to provide a variable air flow rate ranging from about 50 to about 100 L/min.
